(19) United States
(12) Patent Application Publication
(10) Pub. No.: US 2009/0305294 A1
(43) Pub. Date: Dec. 10, 2009

(54) METHOD OF ISOLATING NUCLEIC ACIDS FROM STOOL SAMPLES

(75) Inventors: Robert James, South Australia (AU); Janette Kazenwadel, South Australia (AU)

Correspondence Address: SCULLY SCOTT MURPHY & PRESSER, PC, 400 GARDEN CITY PLAZA, SUITE 300, GARDEN CITY, NY, 11530, US

(73) Assignee: MediMolecular Pty Ltd, South Australia (AU)

(21) Appl. No.: 12/544,821

(22) Filed: Aug. 20, 2009

Related U.S. Application Data
(63) Continuation of application No. 10/545,542, filed on May 18, 2006, now abandoned.

(51) Int. Cl. C12Q 1/68; C07H 1/06

(52) U.S. Cl. 435/6; 536/25.41

(57) ABSTRACT

The present invention relates to a method of isolating a nucleic acid molecule form a biological sample. More particularly, the present invention relates to a method of isolating ribonucleic acid molecule from a biological sample. The method of the present invention is useful in a range of applications including, but not limited to, diagnostic applications and research and development applications, to the extent that the isolation of nucleic acid molecules, and in particular ribonucleic acid molecules, is required. Most particularly, the method of the present invention provides for the isolation of ribonucleic acid molecules which are suitable for analysis by reverse transcriptase-PCR.

### FIELD OF THE INVENTION

The present invention relates to a method of isolating a nucleic acid molecule from a biological sample. More particularly, the present invention relates to a method of isolating a ribonucleic acid molecule from a biological sample. The method of the present invention is useful in a range of applications including, but not limited to, diagnostic applications and research and development applications, to the extent that the isolation of nucleic acid molecules, and in particular ribonucleic acid molecules, is required. Most particularly, the method of the present invention provides for the isolation of ribonucleic acid molecules which are suitable for analysis by reverse transcriptase-PCR.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Adenomas are benign tumours of epithelial origin which are derived from glandular tissue or exhibit clearly defined glandular structures. Some adenomas show recognisable tissue elements, such as fibrous tissue (fibroadenomas), while others, such as bronchial adenomas, produce active compounds giving rise to clinical syndromes. Tumours in certain organs, including the pituitary gland, are often classified by their histological staining affinities, for example eosinophil, basophil and chromophobe adenomas.

Adenomas may become carcinogenic and are then termed adenocarcinomas. Accordingly, adenocarcinomas are defined as malignant epithelial tumours arising from glandular structures, which are constituent parts of most organs of the body. This term is also applied to tumours showing a glandular growth pattern. These tumours may be sub-classified according to the substances that they produce, for example mucus secreting and serous adenocarcinomas, or to the microscopic arrangement of their cells into patterns, for example papillary and follicular adenocarcinomas. These carcinomas may be solid or cystic (cystadenocarcinomas). Each organ may produce tumours showing a variety of histological types, for example the ovary may produce both muconus and cystadenocarcinoma. In general, the overall incidence of carcinoma within an adenoma is approximately 5%. However, this is related to size and although it is rare in adenomas of less than 1 centimetre, it is estimated at 40 to 50% villous lesions which are greater than 4 centimetres. Adenomas with higher degrees of dysplasia have a higher incidence of carcinoma. Once a sporadic adenoma has developed, the chance of a new adenoma occurring is approximately 30% within 26 months.

Colorectal adenomas represent a class of adenomas which are exhibiting an increasing incidence, particularly in more affluent countries. The causes of adenoma, and its shift to adenocarcinoma, are still the subject of intensive research. To date it has been determined that in addition to genetic predisposition, environmental factors (such as diet) play a role in the development of this condition. Most studies indicate that the relevant environmental factors relate to high dietary fat, low fibre and high refined carbohydrates.

Colonic adenomas are localised proliferations of dysplastic epithelium which are initially flat, but with increased growth project from the mucosal forming adenomas. They are classified by their gross appearance as either sessile (flat) or penduculated (having a stalk). While small adenomas (less than 0.5 millimetres) exhibit a smooth tan surface, penduculated adenomas have a head with a cobblestone or lobulated red-brown surface. Sessile adenomas exhibit a more delicate villous surface. Penduculated adenomas are more likely to be tubular or tubulovillous while sessile lesions are more likely to be villous. Sessile adenomas are most common in the cecum and rectum while overall penduculated adenomas are equally split between the sigmoid-rectum and the remainder of the colon.

Screening for neoplasms has generally centred on detecting faecal (stool) occult blood. However, in light of on-going research in relation to the genomic contribution to the onset of such cancers, there has arisen a need to isolate and analyse the nucleic acid molecule population present in relevant biological samples such as stools. To date, such analyses have focused on the isolation of deoxyribonucleic acid (herein referred to as “DNA”) in order to screen for the presence of gene mutations which are thought to be indicative of the onset of a neoplastic condition. Methods for isolating genomic DNA are well known, although there nevertheless exists an ongoing need to develop more efficient methodology.

In terms of the elucidation of the genomic causes of colorectal neoplasms, and in particular adenoma development, the present inventors have previously determined that at the genomic level, such cancers are sometimes marked by an alteration in the expression levels of otherwise normal genes rather than the existence of gene mutations, per se. However, screening for altered gene expression levels necessarily requires the isolation and analysis of ribonucleic acid (herein referred to as “RNA”) rather than DNA.

The isolation of RNA from biological samples is generally recognised as a difficult and inefficient procedure due to the inherent instability of RNA. Further, most available methods focus on the isolation of mRNA via its polyA tail, a technique which is not suitable where one is either seeking to isolate total RNA (for example, mRNA together with primary RNA transcripts) or where the biological environment is such that the mRNA of interest may have undergone some degree of degradation and therefore lost its polyA tail (for example, as is known to occur with respect to stool mRNA). Further, to date it has not been possible to successfully perform reverse transcriptase-PCR (herein referred to as “RT-PCR”) on RNA populations isolated from human stools.

Accordingly, there is a need to develop improved methods for isolating total RNA and, in particular, mRNA which may lack its polyA tail. In this regard, although there have been described methods of isolating RNA from rat stools, due to dietary and other differences, such as differences in gut bacterial flora, digestive enzymes and nutrient absorptive mechanisms, which exist between the rat and the human, these methods are not applicable to the isolation of RNA from human stools. Further, these methods focus on the isolation of polyA mRNA. As detailed hereinbefore, most RNA in stools is degraded and therefore lacks its polyA tail. Accordingly, even if these methods were applicable to the human system, they would not achieve isolation of the total RNA pool. Still further, the RNA product obtained utilising such prior art methodology cannot be analysed by the technique of RT-PCR.

In work leading up to the present invention, the inventors have developed methodology which achieves the isolation of RNA from a human biological sample such as stools. Since this method is not directed to the isolation of RNA based on probing for a polyA tail, the isolation of total RNA is thereby achieved. Further, the RNA isolated in accordance with this method can be successfully amplified utilising RT-PCR. The development of this method now facilitates the routine isolation of total RNA from a biological sample irrespective of its relative state of degradation and thereby, inter alia, provides a means for more accurately and sensitively analysing gene expression levels. The method of the present invention is therefore useful in a range of situations, including but not limited to, as part of a routine diagnostic protocol directed to identifying the onset or risk thereof of colorectal neoplasms which are marked by altered expression levels of unmutated genes detectable in the stools of patients.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides a method for the isolation of a nucleic acid molecule from a biological sample, said method comprising the steps of:
(i) subjecting said biological sample to a protein precipitation step;
(ii) subjecting the soluble component of the biological sample precipitated in accordance with step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the nucleic acid molecule component of said sample; and
(iv) isolating said precipitated nucleic acid molecule.

Another aspect of present invention more particularly provides a method for the isolation of RNA from a biological sample, said method comprising the steps of;
(i) subjecting said biological sample to a protein precipitation step;
(ii) subjecting the soluble component of the biological sample precipitated in accordance with, step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the RNA component of said sample; and
(iv) isolating said precipitated RNA.

In yet another aspect there is provided a method for the isolation of a RNA molecule from human stool, said method comprising the steps of:
(i) subjecting said stool sample to a protein precipitation step;
(ii) subjecting the soluble component of the stool sample precipitated in accordance with step (i) to a chloroform extraction or functionally equivalent extraction;
(iii) contacting the soluble component of the stool sample extracted in accordance with step (ii) with a salt or functional derivative, analogue, equivalent or mimetic thereof together with isopropanol or functional derivative, analogue, equivalent or mimetic thereof and incubating said component for a time and under conditions sufficient to induce precipitation of RNA component of said stool; and
(iv) isolating said precipitated RNA.

Still another aspect of the present invention provides a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample precipitated in accordance with step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation, of the RNA component of said sample; and
(iv) isolating said precipitated RNA.

A further aspect of the present invention provides a method for the isolation RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a tune and under conditions sufficient to induce precipitation of the RNA component of said sample; and
(iv) isolating said precipitated RNA.

Another further aspect of the present invention provides a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with NaCl and/or Na-citrate together with isopropanol or functional derivative, analogue, equivalent or mimetic thereof and incubating said sample for a time and under conditions sufficient to induce precipitation of the RNA component of said sample; and
(iv) isolating said precipitated RNA.

In yet another further aspect there is provided a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with 50% v/v of 1.2M NaCl and 0.8M Na-citrate pH 7.0 or functional derivative, analogue, equivalent or mimetic thereof together with 50% v/v isopropanol or functional derivative, analogue, equivalent or mimetic thereof and incubating said sample for a time and under conditions sufficient to induce precipitation of the RNA component of said sample; and
(iv) isolating said precipitated RNA.

In still yet another further aspect the present invention provides a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with 50% v/v of 1.2M NaCl and 0.8M Na-citrate pH 7.0 or functional derivative, analogue, equivalent or mimetic thereof together with 50% v/v isopropanol or functional derivative, analogue, equivalent or mimetic thereof and incubating said sample for at least 60 minutes at or about −200° C.; and
(iv) isolating said precipitated RNA.

Still another aspect of the present invention contemplates nucleic acid molecules isolated in accordance with the method of the present invention.

Preferably, said nucleic acid molecules are RNA molecules.

Yet another aspect of the present invention is directed to a method of isolating RNA from a biological sample, which RNA is suitable for analysis by RT-PCR, utilising the RNA isolation methodology hereinbefore defined.

The present invention should be understood to extend to the use of the subject nucleic acid isolation methodology in the diagnosis and/or monitoring of conditions characterised by aberrant nucleic acid expression and/or in other screening methods which require the isolation of nucleic acid populations, in particular RNA populations, for analysis.

Preferably, said isolated RNA is suitable for RT-PCR analysis and said condition is colorectal adenoma development.

The present invention still further extends to the use of nucleic acid molecules isolated in accordance with the method of the present invention in the treatment and/or diagnosis or monitoring of patients. Accordingly, another aspect of the present invention contemplates a pharmaceutical composition comprising nucleic acid molecules isolated according to the method of the present invention together with one or more pharmaceutically acceptable carriers and/or diluents.

Yet another aspect of the present invention is directed to a kit for facilitating the isolation of a nucleic acid molecule from a biological sample said kit comprising compartments adapted to contain any one or more of protein extraction reagents, chloroform extraction reagents, salt, isopropanol and means for isolating the precipitated nucleic acid molecule. Further compartments may also be included, for example, to receive biological samples.

### BRIEF DESCRIPTION OF THE DRAWINGS

FIG. 1 is an image of β-actin RT-PCR from adult human stool.

FIG. 2 is an image of β₂-microglobulin and heat shock protein RT-PCR from adult human stool samples.

### DETAILED DESCRIPTION OF THE INVENTION

The present invention is predicated, in part, on the determination that total RNA can be routinely and efficiently isolated from a human biological sample provided that the isolation methodology incorporates, subsequently to an initial protein precipitation step, a chloroform extraction of the soluble material derived from said protein precipitation step followed by precipitation, in the presence of both isopropanol and a high salt concentration, of the RNA component derived therefrom. The development of an RNA isolation method which is based on RNA precipitation, rather than the detection and isolation of polyA+ mRNA transcripts, now facilitates the isolation and analysis of total RNA, irrespective of its state of degradation. The method of the present invention is therefore particularly useful for isolating RNA from biological samples in which partial degradation of mRNA can quickly occur, such as occurs in stools. The present invention is also unique in that it enables the RT-PCR analysis of RNA extracted from stool, this being an aspect of analysis which has not been feasible to date, despite this being a commonly recognised difficulty in respect of which a solution has long, but unsuccessfully, been sought.

Accordingly, one aspect of the present invention provides a method for the isolation of a nucleic acid molecule from a biological sample, said method comprising the steps of:
(i) subjecting said biological sample to a protein precipitation step;
(ii) subjecting the soluble component of the biological sample precipitated in accordance with step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the nucleic acid molecule component of said sample; and
(iv) isolating said precipitated nucleic acid molecule.

Reference to “nucleic acid molecule” should be understood as a reference to both DNA and RNA or derivatives or analogues thereof. Preferably, the subject nucleic acid molecule is RNA. In this regard, the subject RNA should be understood to encompass all forms of RNA including, but not limited to, primary RNA transcripts, messenger RNA, transfer RNA and ribosomal RNA. Nucleic acid molecules which are isolated in accordance with the method of the present invention may be of any origin including naturally occurring or may have been recombinantly or synthetically produced and introduced into a subject for any one or more of a number of reasons including, but not limited to, for the purpose of gene therapy or gene transfer procedures or for use as an in vivo marker or targeting means.

The present invention therefore more particularly provides a method for the isolation of RNA from a biological sample, said method comprising the steps of:
(i) subjecting said biological sample to a protein precipitation step;
(ii) subjecting the soluble component of the biological sample precipitated in accordance with step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the RNA component of said sample; and
(iv) isolating said precipitated RNA.

Reference to a “biological sample” should be understood as a reference to any sample of biological material derived from an animal such as, but not limited to, mucus, stool, urine, biopsy specimens and fluid which has been introduced into the body of an animal and subsequently removed such as, for example, the saline solution extracted from the lung following lung lavage or the solution retrieved from an enema wash. The biological sample which is tested according to the method of the present invention may be tested directly or may require some form of pre-treatment prior to testing. For example, a biopsy or stool sample may require homogenisation prior to testing. Further, to the extent that the biological sample is not in a soluble form (for example it may be a solid, semi-solid or a dehydrated sample) it may require the addition of a reagent, such as a buffer, to mobilise the sample. It should be further understood that the sample which is the subject of testing may be freshly isolated or it may have been isolated at an earlier point in time and subsequently stored or otherwise treated prior to testing. For example, the sample may have been collected at an earlier point in time and freeze-dried or otherwise preserved in order to facilitate its transportation to the site of testing. In another example, to the extent that the subject sample is a stool sample, in one embodiment of the method of the invention, the sample is stored for 24-48 hours, at room temperature in a solution of guanidine thiocyanate and Na-citrate. Without limiting the present invention in any way, guanidine thiocyanate is a chaotropic agent which denatures protein. In this context, the denaturation of the stool sample's protein component provides an aid to the efficiency of the initial protein precipitation step which the sample will undergo in accordance with the method disclosed herein.

Preferably, said biological sample is a stool sample.

The term “animal” as used herein includes a human, primate, livestock animal (e.g. sheep, pig, cow, horse, donkey), laboratory test animal (e.g. mouse, rat, rabbit, guinea pig), companion animal (e.g. dog, cat), captive wild animal (e.g. fox, kangaroo, deer), aves (e.g. chicken, geese, duck, emu, ostrich), reptile or fish. Preferably, the subject animal is a human.

According to this preferred embodiment, there is provided a method for the isolation of a RNA molecule from human stool, said method comprising the steps of:
(i) subjecting said stool sample to a protein precipitation step;
(ii) subjecting the soluble component of the stool sample precipitated in accordance with step (i) to a chloroform extraction or functionally equivalent extraction;
(iii) contacting the soluble component of the stool sample extracted in accordance with step (ii) with a salt or functional derivative, analogue, equivalent or mimetic thereof together with isopropanol or functional derivative, analogue, equivalent or mimetic thereof and incubating said component for a time and under conditions sufficient to induce precipitation of the RNA component of said stool; and
(v) isolating said precipitated RNA.

The method of the present invention is predicated on the determination that efficient isolation of total RNA from a biological sample is achievable where the sample is subjected to certain specific extraction steps which are sequentially performed subsequently to an initial protein precipitation step. In this regard, reference to “precipitation” should be understood as a reference to rendering a molecule insoluble. The means by which the subject molecule is rendered insoluble is generally herein referred to as an “extraction” means. In this regard, reference to “extraction” should be understood as a reference to the separation of a molecule from a group of molecules by selective solubility.

As detailed hereinbefore, the biological sample which is subjected to the method of the present invention may be a fresh sample or a stored sample and may have undergone some form of pre-treatment prior to its subjection to the method herein described. For example, in one embodiment the sample is incubated with guanidine thiocyanate and Na-citrate for the purpose of increasing the efficacy of denaturation of the protein component of the subject biological sample.

The inventors have determined that inefficiencies which have previously been observed in RNA isolation protocols which are based on precipitation principles, rather than polyA+ mRNA probing, can be overcome where a specific sequence of extraction steps is performed subsequently to an initial protein precipitation step. In this regard, reference to “subjection” of a biological sample to a “protein precipitation step” in point (i) of the method disclosed herein should be understood as a reference to the application of any suitable protein precipitation method to the biological sample. Methods of precipitating protein based on the principles of selective solubility are well known to those of skill in the art. In this regard, it should be understood that it is not necessary that the subject method precipitates the protein component of the biological sample in its entirety since the subsequently applied chloroform extraction steps of step (ii) will also act to extract residual protein contamination from the sample. It is well within the skills of the person of ordinary skill in the art to select a suitable protein precipitation protocol for use in a given situation. For example, one might consider the suitability of a given method relative to the biological sample type to which it is to be applied. Examples of protein extraction methodologies include, but are not limited to, phenol extraction. By “phenol extraction” is meant a protein extraction method which utilises phenol as the active agent. Methods for utilising phenol in this manner would be well known to the person of skill in the art and are detailed in standard texts such as the Sambrook et al Laboratory Manual (1989).

According to this preferred embodiment, the present invention therefore more particularly provides a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample precipitated in accordance with step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the RNA component of said sample; and
(v) isolating said precipitated RNA.

Preferably, said biological sample is a stool sample and still more preferably a human stool sample.

Reference to a “functionally equivalent extraction step” in the context of a phenol extraction should be understood as a reference to an extraction step which utilises, as the active agent, a functional derivative, analogue, equivalent or mimetic of phenol or a method which utilises a non-phenol molecule as the active agent, but which molecule nevertheless achieves the same objective as phenol, being the denaturation and precipitation of proteins and nucleic acids. It should also be understood that to the extent that a non-phenol molecule is utilised, the denaturation and precipitation of the subject nucleic acid molecules may be achieved utilising two or more active agents.

Preferably, the subject phenol extraction step is performed utilising substantially 10% v/v of 2M NaOAc pH 4.0 (calculated relative to the volume of biological sample to be extracted) and a substantially equal volume of acid-phenol/CHCl3, at a ratio of 5:1.

Following the initial protein precipitation step, the soluble component remaining thereafter is subjected to a chloroform extraction step. Preferably, this chloroform extraction step utilises chloroform alone. By “soluble component” is meant the population of molecules, derived from the biological sample, which have remained in solution subsequently to the phenol extraction step. Without limiting the present invention in any way, these molecules will include the nucleic acid molecule population which is the subject of isolation and may additionally include some protein molecules (possibly denatured) or other contaminants which were not precipitated during the phenol extraction step.

Preferably the soluble component is isolated prior to its subjection to a chloroform extraction step. This is most easily achieved by centrifugation of the phenol extracted sample such that any precipitated molecules are pelleted, thereby facilitating decanting of the aqueous phase of the phenol extracted sample, which aqueous phase comprises the soluble component, that is, the population of molecules which have remained in solution. It should be understood that any other method which renders the soluble component suitable for subjection to a chloroform extraction step may also be utilised.

Still without limiting the present invention in any way, chloroform is also an agent which is not water soluble and acts to precipitate residual phenol left behind after the acid-chloroform extraction. Accordingly, this is a step which, to date, has been optionally used in some prior art RNA extraction protocols. However, without limiting the present invention to any one theory or mode of action, it has been surprisingly determined that a pure chloroform extraction step can achieve removal of contaminants which act to inhibit RT-PCR analysis of the RNA product which is ultimately isolated. Accordingly, this finding has now facilitated the development of a method which has been long sought after, but, to date, has been unobtainable. Preferably, the chloroform extraction step is performed utilising a volume of chloroform which is substantially equal to the volume of the soluble component to which it is introduced. It should be understood that subject to use of the preferred volumed detailed herein, the performance of a chloroform extraction step would be well known to the person of ordinary skill in the art. In this regard, to the extent that the chloroform extraction results in the formation of a visible interface, an additional chloroform extraction step is preferably performed. Reference to an extraction step which is “functionally equivalent” to the subject chloroform extraction step should be understood to have an analogous meaning to the phenol related functionally equivalent extraction step.

The present invention therefore preferably provides a method for the isolation RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the RNA component of said sample; and
(iv) isolating said precipitated RNA.

Preferably, said biological sample is a stool sample and still more preferably a human stool sample.

As detailed hereinbefore, recovery of the soluble component may be achieved by any suitable method, which methods would be well known to the person of skill in the art. In this regard, reference to the “soluble component” remaining subsequently to chloroform extraction should be understood to have a meaning analogous to that hereinbefore provided in relation to the soluble component recited in relation to step (ii) of the subject method. In accordance with the method of the present invention, the soluble component remaining after the chloroform extraction step is subjected to both a high salt concentration and the introduction of isopropanol or functional derivative, analogue, equivalent or mimetic thereof prior to incubation of this solution for a time and under conditions sufficient to induce precipitation of the nucleic acid population which is present in the solution. Reference to “salt” should be understood as a reference to any suitable form of sodium or functional derivative, analogue, equivalent or mimetic thereof including both soluble and crystalline forms. In a preferred embodiment, said salt is NaCl and/or Na-citrate, lithium and/or potassium.

The present invention therefore still more preferably provides a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with NaCl and/or Na-citrate together with isopropanol or functional derivative, analogue, equivalent or mimetic thereof and incubating said sample for a time and under conditions sufficient to induce precipitation of the RNA component of said sample; and
(iv) isolating said precipitated RNA.

Preferably, said biological sample is a stool sample and still more preferably a human stool sample.

Reference to the phrase “together with” should be understood to mean that the salt and isopropanol are both present in the subject soluble component during at least part of the incubation phase. The salt and isopropanol may be co-administered or may be administered in any suitable sequential order. Reference to “contacting” should be understood to mean any form of exposure of at least part of the subject soluble component to the salt and isopropanol. In a preferred embodiment, the salt is administered subsequently to the isopropanol.

In a preferred embodiment, the inventors have determined that this step of the present invention is optimally performed utilising substantially 50% v/v isopropanol followed by addition to the subject solution of substantially 50% v/v of 1.2M NaCl/0.8M Na-citrate pH 7.0 or functional derivative, equivalent, analogue or mimetic thereof. Volume percentages are calculated relative to the volume of the soluble component.

Accordingly, there is most preferably provided a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with substantially 50% v/v of 1.2M NaCl and 0.8M Na-citrate pH 7.0 or functional derivative, analogue, equivalent or mimetic thereof together with substantially 50% v/v isopropanol or functional derivative, analogue, equivalent or mimetic thereof and incubating said sample for a time and under conditions sufficient to induce precipitation of the RNA component of said sample; and
(iv) isolating said precipitated RNA.

Preferably, said biological sample is a stool sample and still more preferably a human stool sample.

Reference to “phenol”, “chloroform”, “salt”, “NaCl”, “Na-citrate” and “isopropanol” should be understood as a reference to all forms of these molecules (such as all isomeric forms) and, to the extent that it is not specified, to functional derivatives, analogues, equivalents and mimetics thereof. These molecules may take any suitable physical form including, but not limited to, crystalline, desiccated or soluble forms.

“Functional derivatives” include fragments, parts, portions, mutants, and mimetics from natural, synthetic or recombinant sources including fusion proteins exhibiting any one or more of the functional activities of the subject molecule. To the extent that the subject molecule is a protein, derivatives may be derived from insertion, deletion or substitution of amino acids. Ammo acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences including fusions with other peptides, polypeptides or proteins.

Derivatives include fragments having particular parts of the entire molecule fused to peptides, polypeptides ox other proteinaceous or non-proteinaceous molecules.

Reference to “derivatives” should also be understood to include reference to analogs. Analogs contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Examples of side chain modifications contemplated by the present invention include modifications of ammo groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of ammo groups with succinic anhydride and tetrahydrophthalic anhydride; and pyridoxylation of lysine with pyridoxal-5-phosphate fallowed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide. Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, fox example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-τ-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilise 3D conformations, using homo-bifunctianal crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-Hydroxysuccinimide and another group specific-reactive moiety.

“Functional equivalents” of the subject molecules include, for example, chemical equivalents exhibiting any one or more of the functional activities of the subject molecule. Chemical equivalents may, for example, share certain conformational similarities. Alternatively, chemical equivalents may be specifically designed to mimic certain physiochemical properties of the subject molecule. Chemical equivalents may be chemically synthesised or may be detected, for example, by natural product screening.

The incubation step detailed in point (iii) of the method defined herein is preferably performed at a temperature of less than 0° C., preferably less than −20° C., more preferably less than −50° C., still more preferably less than −80° C., yet more preferably less than −100° C., still more preferably less than −120° C., yet still more preferably less than −150° C., more preferably less than −180° C. and most preferably, at or about −200° C. for at least 10 minutes, preferably 30 minutes and most preferably at least 60 minutes.

The present invention therefore most preferably provides a method for the isolation of RNA from a biological sample, said method comprising the steps of:
(i) subjecting said biological sample to a protein precipitation step, wherein said protein precipitation is induced and/or otherwise facilitated utilising a phenol extraction step or functionally equivalent extraction step;
(ii) subjecting the soluble component of the biological sample extracted in accordance with step (i) to a chloroform extraction or functionally equivalent extraction, wherein said chloroform is utilised at a volume substantially equal to the volume of said soluble component;
(iii) contacting the soluble component of the biological sample extracted in accordance with step (ii) with substantially 50% v/v of 1.2M NaCl and 0.8M Na-citrate pH 7.0 or functional derivative, analogue, equivalent or mimetic thereof together with substantially 50% v/v isopropanol or functional derivative, analogue, equivalent or mimetic thereof and incubating said sample for at least 60 minutes at or about −200° C.; and
(iv) isolating said precipitated RNA.

Preferably, said biological sample is a stool sample and more preferably a human stool sample.

Without limiting the present invention to any one theory or mode of action, the isopropanol precipitation step hereinbefore described facilitates precipitation of nucleic acid molecules, and in particular RNA. As detailed hereinbefore, recovery of the precipitated nucleic acid molecule may be achieved by any suitable technique which would be well known to the person of suitable skill in the art. For example, the precipitated nucleic acid molecule may be pelleted utilising ultracentrifugation.

Subsequently to its isolation, the RNA precipitate may be subjected to any number of further washing and/or precipitation steps as determined to be necessary by the person of skill in the art. Such additional steps may be performed, for example, to remove trace non-nucleic acid contaminants. The steps which one may seek to perform are well known to those skilled in the art and determination of the necessity and/or suitability of their application would involve no more than routine analysis. For example, in one embodiment, the precipitate which is recovered subsequently to the isopropanol incubation step described herein may be washed in alcohol (for example, EtOH or functional derivative, analogue, equivalent or mimetic thereof), further incubated in 2.5M LiCl at or about −200° C. for approximately 30 minutes and the precipitate derived therefrom washed in still further alcohol. Without limiting the method of the invention in any way, these washing and additional precipitation steps are standard methodologies which one may seek to employ.

In this regard, it should be understood that the present invention may optionally comprise one or more additional steps. The person of skill in the art may elect to introduce additional steps which may be particularly useful with respect to a given situation. For example, and as detailed hereinbefore, the means of isolating and preparing a biological sample for use in accordance with the method of the present invention will likely vary according to the nature of the sample itself. Suitable preparative protocols could be routinely determined by the person of skill in the art based on common knowledge and experience. In another example, in certain circumstances the person of skill in the art may elect to introduce a purification step such as a cesium chloride separation step in order to separate double versus single stranded nucleic acid molecules. Such techniques may be particularly suitable for separating single stranded RNA from double stranded DNA. The applicability of such a step would likely depend on the outcome to be achieved and on the nature of the biological sample from which the nucleic acid molecules are being isolated. In yet another example, although the preferred method is to isolate RNA, to the extent that it may be desirable to isolate both DNA and RNA, it may be necessary to rupture cellular nuclei which are present in a biological sample, thereby facilitating access to genomic DNA. Still further, in some circumstances, it may be desirable to pass a sample of RNA isolated in accordance with the method of the present invention through an oligo dT column in order to facilitate the analysis of polyA+ RNA.

It should be understood that these modifications are described by way of example only and are not intended to limit the scope of the routine modifications which one may introduce to the method of the invention as defined herein, which modified methods should be understood to fall within the scope of the isolation methodology defined herein.

Detection and analysis of the nucleic acid molecules isolated in accordance with the method of the present invention may be performed by any suitable means. In this regard, it should be understood that although the nucleic acid molecules isolated in accordance with the method of the present invention are highly enriched, depending on the nature of the starting biological sample, there may nevertheless be present some trace contamination of non-nucleic acid material. Accordingly the term “isolating” should be understood to encompass both purifying out a nucleic acid population and enriching for the subject population.

Still another aspect of the present invention contemplates nucleic acid molecules isolated in accordance with the method of the present invention.

Preferably, said nucleic acid molecules are RNA molecules.

The development of the method of the present invention now facilitates the routine yet highly efficient isolation, from a biological sample, of the nucleic acid population and, in particular, the RNA population. Accordingly, the present invention has particular significance with respect to diagnostic procedures and research and development applications which require isolation of whole RNA or degraded mRNA populations. As detailed hereinbefore, this is of particular relevance where one is seeking to analyse RNA expression in biological samples which rapidly degrade RNA, thereby rendering ineffective polyA+ tail based probing strategies. Most significantly, the method of the present invention facilitates the isolation of an RNA population which is suitable for analysis by RT-PCR. To date, this form of analysis has not been possible on RNA samples isolated from stool. Accordingly, the development of the present invention has now achieved a long searched for and highly sought after outcome.

It should therefore be understood that yet another aspect of the present invention is directed to a method of isolating RNA from a biological sample, which RNA is suitable for analysis by RT-PCR, utilising the RNA isolation methodology hereinbefore defined.

Reference to RNA being “suitable for analysis by RT-PCR” should be understood as a reference to at least a proportion of the isolated RNA sample being capable of amplification by RT-PCR.

Still another aspect of the present invention should be understood to extend to the use of the subject nucleic acid isolation methodology in the diagnosis and/or monitoring of conditions characterised by aberrant nucleic acid expression and/or in other screening methods which require the isolation of nucleic acid populations, in particular RNA populations, for analysis.

Preferably, said isolated RNA is suitable for RT-PCR analysis. Even more preferably, said condition is colorectal adenoma development.

The present invention still further extends to the use of nucleic acid molecules isolated in accordance with the method of the present invention in the treatment of patients and/or diagnosis or monitoring of disease conditions. Accordingly, another aspect of the present invention, contemplates a pharmaceutical composition comprising nucleic acid molecules isolated according to the method of the present invention together with one or more pharmaceutically acceptable carriers and/or diluents.

Yet another aspect of the present invention is directed to a kit for facilitating the isolation, of a nucleic acid molecule from a biological sample said kit comprising compartments adapted to contain any one or more of protein, extraction reagents, chloroform extraction reagents, salt, isopropanol and means for isolating the precipitated nucleic acid molecule. Further compartments may also be included, for example, to receive biological samples.

The present invention is further described by the following non-limiting examples:

### EXAMPLE 1

### Protocol for Extraction of RNA From Stool Samples

### Collection of Samples

Approximately 2-5 g samples were placed in 20 ml of a solution of 4M guanidine thiocyanate and 20 mM Na-citrate, pH 7.0, dispersed by shaking and stored at room temperature for 24-48 hours before processing. RNA has also been extracted from samples stored at 4° C. for two weeks.

### RNA Extraction

1. Ensure that sample is dispersed as completely as possible
2. Spin at 3000 rpm for 10 minutes
3. Transfer supernatant to fresh tube and homogenise
4. Add 0.1 volumes 2M NaOAc pH4.0 followed by an equal volume of acid-phenol/CHCl3 (5:1)
5. Vortex well and incubate on ice for 20 min
6. Spin at 10000 rpm for 20 min at 4° C.
7. Recover aqueous phase and extract with an equal volume of CHCl3. (If a visible interface forms perform an additional chloroform extraction)
8. Recover aqueous phase and add 0.5 volume of isopropanol followed by an equivalent volume of 1.2M NaCl/0.8M Na-citrate pH7.0. (That is if the recovered aqueous volume is 1 ml men add 0.5 ml of isopropanol and 0.5 ml of 1.2M NaCl/0.8M citrate)
9. Precipitate at −200° C. for at least 60 min
10. Spin at 10000 rpm for 10 min at 4° C.
11. Rinse pellet with 0.2 ml 75% EtOH
12. Resuspend in sterile dH₂O and spin for 5 min in an eppendorf centrifuge at 10000 rpm for 10 min
13. Discard pellet and adjust the supernatant to a final concentration of 2.5M LiCl
14. Precipitate at −200° C. for at least 30 min
15. Centrifuge sample for 20 min at 4° C.
16. Wash pellet with 75% EtOH
17. Resuspend in sterile dH₂O

### EXAMPLE 2

### Results From the Extraction of RNA From Stool Samples

RNA has been isolated from the stools of both children and adults who are subject to a wide range of diets. Ten individual samples were analysed.

Prior to the advent of the present method, there were reports detailed in the literature of RNA isolation methods directed to isolating RNA from human stools. However, Reverse Transcriptase-PCR had not previously been successfully performed on this material.

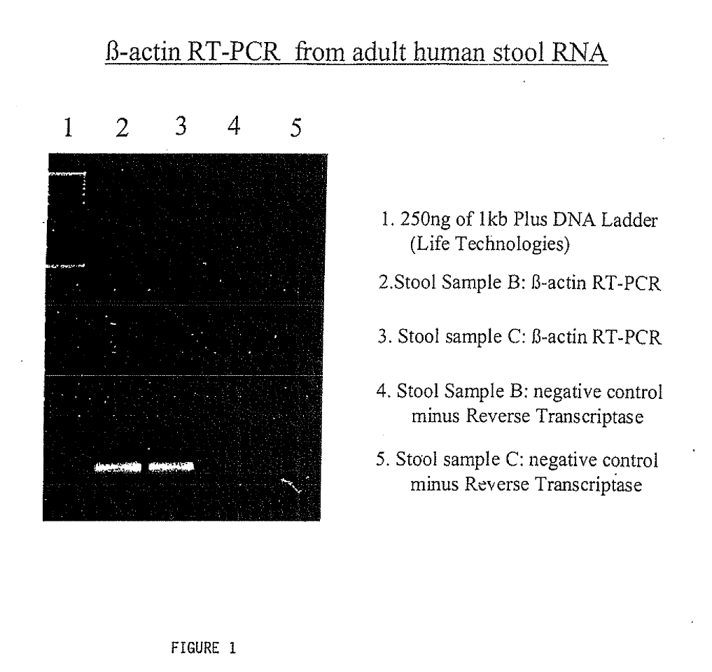

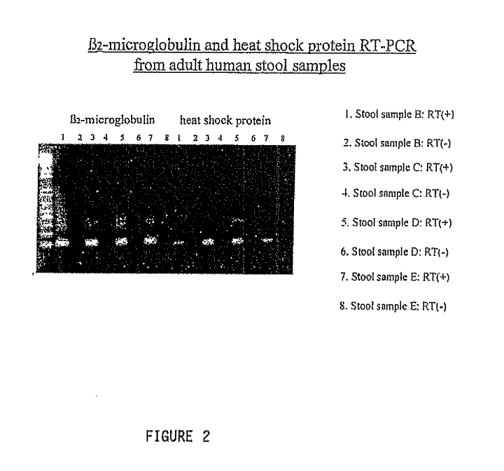

The RNA isolated in accordance with these examples has been successfully subjected to Reverse Transcriptase-PCR. FIGS. 1 and 2 demonstrate these results. Specifically, with respect to the β-actin experiment, the correct sized product only appears in the first gel lanes 2 and 3. The weak bands in lanes 3 and 4 represent background. Similarly, the strong bands evident in both the β2-microglobulin and heat shock protein related image represent the expected produce size.

Those skilled in the art will appreciate that the invention described herein is susceptible to variations and modifications other man those specifically described. It is to be understood that the invention includes all such variations and modifications. The invention also includes all of the steps, features, compositions and compounds referred to or indicated in this specification, individually or collectively, and any and all combinations of any two or more of said steps or features.

### Bibliography

Sambrook et at; “Molecular Closing: a laboratory manual”, second edition, Cold Spring Harbour Laboratory Press, Cold Spring Harbour, N.Y., 1989.

1. A method for the isolation of a nucleic acid molecule from a biological sample, said method comprising the steps of:
(i) subjecting said biological sample to a protein precipitation step;
(ii) subjecting the soluble component of the biological sample precipitated in accordance with step (i) to a chloroform extraction or functionally equivalent extraction;
(iii) contacting the soluble component of the biological sample extracted in accordance with step (ii) with a salt or functional derivative, analogue, equivalent or mimetic thereof together with isopropanol or functional derivative, analogue, equivalent or mimetic thereof and incubating said sample for a time and under conditions sufficient to induce precipitation of the nucleic acid molecule component of said sample; and
(iv) isolating said precipitated nucleic acid molecule.

2. The method according to claim 1 wherein said nucleic acid molecule is RNA.

3. The method according to claim 2 wherein said isolated RNA is suitable for analysis by reverse-transcriptase PCR.

4. The method according to claim 2 or 3 wherein said biological sample is a stool sample.

5. The method according to any one of claims 1-4 wherein said chloroform of step (ii) is utilised at a volume substantially equal to said soluble component.

6. The method according to any one of claims 1-4 wherein said soluble component of step (iii) is extracted with NaCl and/or Na-citrate together with isopropanol.

7. The method according to claim 6 wherein said NaCl and/or Na-citrate is 50% v/v of 1.2 M NaCl and 0.8 M Na-citrate pH 7.0 and said isopropanol is 50% v/v isopropanol.

8. The method according to claim 7 wherein the sample of step (ii) is incubated for at least 30 minutes, and preferably 60 minutes, at below −100° C.

9. The method Hoarding to claim 8 wherein said sample is incubated for at least 30 minutes, and preferably 60 minutes, at below −150° C.

10. The method according to claim 9 wherein said sample is incubated for at least 30 minutes, and preferably 60 minutes, at or about −200° C.

11. The method according to any one of claims 1-4 wherein said chlorform of step (ii) is utilised at a volume substantially equal to said soluble component and said soluble component of step (iii) is extracted with NaCl and/or Na-citrate together with isopropanol.

12. The method according to claim 11 wherein said NaCl and/or Na-citrate is 50% v/v of 1.2 M NaCl and 0.8 M Na-citrate pH 7.0 and said isopropanol is 50% v/v isopropanol.

13. The method according to claim 12 wherein the sample of step (iii) is incubated for at least 30 minutes, and preferably 60 minutes, at below −100° C.

14. The method according to claim 13 wherein said sample is incubated for at least 30 minutes, and preferably 60 minutes, at below −150° C.

15. The method according to claim 14 wherein said sample is incubated for at least 30 minutes, and preferably 60 minutes, at or about −200° C.

16. The method according to any one of claims 5-15 wherein said protein precipitation of step (i) is a phenol extraction step or functionally equivalent extraction step.

17. Use of the method of any one of claims 1-16 in any one or more screening methods, which methods are characterised by the isolation of a nucleic acid population.

18. Use according to claim 17 wherein said screening method is the diagnosis and/or monitoring of conditions characterised by aberrant nucleic acid expression.

19. Use according to claim 18 wherein said condition is colorectal adenoma development.

20. Use according to any one of claims 17-19 wherein said nucleic acid population is RNA.

21. A kit when used in accordance with the method of any one of claims 1-16 said kit comprising compartments adapted to contain any one or more of protein extraction reagents, chloroform extraction reagents, salt, isopropanol and means for isolating the precipitated nucleic acid molecule.

22. A method according to any one of claims 1-16 or a use according to any one of claims 17-20 or a kit according to claim 21 substantially as hereinbefore described with reference to the Figures and/or Examples.
